(19) United States
(12) Patent Application Publication
(10) Pub. No.: US 2005/0245494 A1
(43) Pub. Date: Nov. 3, 2005

(54) Methods to treat one or all of the defined etiologies of female sexual dysfunction

(75) Inventors: Ronald J. Thompson, Fort Thomas, KY (US); James M. Thompson, Cincinnati, OH (US); Justin R. Thompson, Cincinnati, OH (US)

Correspondence Address: Donald N. Halgren, 35 Central Street, Manchester, MA, 01944, US

(73) Assignee: 40 J's LLC

(21) Appl. No.: 11/176,546

(22) Filed: Jul. 7, 2005

Related U.S. Application Data
(63) Continuation-in-part of application No. 10/989,978, filed on Nov. 16, 2004.
Application No. 10/989,978 is a continuation-in-part of application No. 10/803,148, filed on Mar. 17, 2004.
Application No. 10/989,978 is a continuation-in-part of application No. 10/731,692, filed on Dec. 9, 2003.
Application No. 10/731,692 is a division of application No. 10/004,091, filed on Oct. 23, 2001, now Pat. No. 6,702,733.
Application No. 10/004,091 is a continuation of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 31/56; A61K 31/557; A61K 31/045

(52) U.S. Cl. 514/171; 514/729

(57) ABSTRACT

The U.S. Food and Drug Administration defines the four separate components of Female Sexual Dysfunction (FSD) to be decreased sexual desire, decreased sexual arousal, dyspareunia (pain with intercourse), and persistent difficulty in achieving or inability to achieve orgasm (anorgasmia). To establish a diagnosis of FSD, these components must be associated with personal distress, as determined by the affected woman. A topical combination of menthol/L-arginine and the specific 5-PDE inhibitor vardenafil can be used to treat any of the defined components, or a combination of two or more of the defined components of FSD, as a safe, first-line therapy for FSD.

This invention relates to arrangements for the treatment or alleviation of the symptoms associated with Female Sexual Dysfunction, particularly, to the topical application of specialized stimulatory medicaments. This application is a continuation-in-part application of co-pending U.S. patent applications Ser. No. ______, filed ______ (Thompson 18), and co-pending U.S. patent application Ser. Nos. ______ filed ______ (Thompson 20), ______, filed ______ (Thompson 19), 11/014,429, filed 16 Dec. 2004 (Thompson 17), 10/989,978, filed 16 Nov. 2004 (Thompson 15), 10/651,615, filed 30 Aug. 2003 (Thompson 11A), and 10/407,748, filed 3 Sep. 2003 (Thompson 11), which are continuation-in-part of applications of co-pending U.S. patent application Ser. Nos. 10/803,148 (Thompson 4C) and. 10/731,692 (Thompson 4B), both filed 9 Dec. 2003 which are divisional applications of U.S. patent application Ser. No. 10/004,091 (Thompson 4A), filed 23 Oct. 2001, now U.S. Pat. No. 6,702,733 issued 9 Mar. 2004, which is a continuation of application Ser. No. 09/520,110 (Thompson 4), filed 7 Mar. 2000, now U.S. Pat. No. 6,322,493 which is a continuation-in-part of application Ser. No. 09/469,959 (Thompson 3) filed on 21 Dec. 1999, which is a continuation-in-part of application Ser. No. 09/414,250 (Thompson 2), filed on 7 Oct. 1999, now U.S. Pat. No. 6,224,541 which is a continuation-in-part of application Ser. No. 09/340,227 (Thompson 1), filed on 1 Jul. 1999, now U.S. Pat. No. 6,179,775 each of which are incorporated herein by reference in their entirety.

### FIELD OF THE INVENTION

### BACKGROUND OF THE INVENTION

In May 2000 the U.S. Department of Health and Human Services, the Food and Drug Admistration, and the Center for Drug Evaluation and Research issued a Guidance for Industry titled “Female Sexual Dysfunction: Clinical Development of Drug Products for Treatment.” Within this guidance, the FDA defined four recognized components of Female Sexual Dysfunction:
decreased sexual desire
decreased sexual arousal
dyspareunia (pain with intercourse)
persistent difficulty in achieving or inability to achieve orgasm (anorgasmia).

The FDA further states that “to establish a diagnosis of FSD, these components must be associated with personal distress, as determined by the affected woman.” The guidance then describes subsets of women to be studied to establish efficacy of a test medication over placebo in double blind clinical trials.

In December 2004, Procter and Gamble Pharmaceuticals presented to the FDA the results of their proprietary 300 microgram transdermal testosterone patch against placebo in surgically induced menopause and naturally occurring menopause. The outcome of the Procter and Gamble Pharmaceutical FDA presentation were one, the transdermal testosterone patch was effective in increasing desire and satisfying sexual experiences for women, two, the transdermal testosterone patch was not established as safe because of the conversion of testosterone into estrogen and estrogen's implication in breast cancer, stroke, and heart disease, and three, the transdermal testosterone patch was not proven effective for any other group of women, other than menopausal women who were diagnosed as suffering FSD because of decreased sexual desire. The FDA, through its Physician's Advisory Panel, made it quite clear that if efficacy was established for a specific medication, in a specific subset of women, with a specific diagnostic component of FSD (i.e. decreased sexual desire) the indication granted on approval of the medication would be limited only to the single component of FSD, not all four components of FSD. Therefore, the testosterone transdermal patch could not be indicated for young women on contraceptive medications to increase their desire, because this specific subset of women had not been included within the efficacy studies.

U.S. Pat. Nos. 6,179,775; 6,224,541; 6,322,493; and 6,702,733 describe a topical clitorally applied combination of menthol and L-arginine to enhance a woman's sexual arousal and to help a woman achieve an orgasm. Clinical studies that substantiate these teachings can be found in patent applications ______ and ______. Unnumbered patent application titled “Topical menthol, or related cooling compound to induce lubrication” describes of the use of menthol topically applied to the clitoris to evoke a reflex vaginal lubrication. Lubrication insufficiency alone, or associated with atrophic vaginitis and decreased vaginal elasticity are the etiologies of dyspareunia. Although clinical efficacy data are unavailable for the treatment of dyspareunia component of FSD with menthol, the overall lubrication data from clinical studies reported in “Topical menthol, or related cooling compound to induce lubrication,” gives confidence that dyspareunia can be effectively treated with menthol/L-arginine combination topically applied to the clitoris. Therefore, three of the components of FSD as defined in the Guidance to Industry can be treated with menthol/L-arginine applied to the clitoris. These three components are: decreased sexual arousal, dyspareunia, and persistant difficulty in achieving or the inability to achieve orgasm.

Clinical studies on menthol/L-arginine in combination with topical sildenafil, as described in patent application Ser. No. 10/803,148 or in combination with the more potent 5 PDE inhibitor vardenafil, described in U.S. patent application number (CSC-V), showed even greater effect for treatment of arousal and orgasmic disorder. To be an effective and safe treatment for all four of the components of FSD, the combination of menthol/L-arginine/5-PDE inhibitor must be able to increase desire.

Decreased Sexual Desire

Decreased Sexual desire has two interrelated and overlapping etiologies. The first is physiological, and the other psychological. Although the transdermal testosterone patch Intrinsa described in U.S. Pat. No. 6,583,129 proved to increase desire and more satisfying sexual experiences over placebo, a very interesting finding within the clinical trials underscores the importance of the psychological component of decreased sexual desire. Thirty six percent of the women who used the placebo during the Intrinsa clinical trials reported that the placebo patch not only increased desire, but they also asked to continue using the placebo patch. The patients did not know the patches were placebo because this was a double blind placebo controlled study of over eleven hundred women, over six months of usage. As reported in the 10 Jun. 2005 Science, “[i]ronically, the drug trials themselves suggest that some women may not need desire-boosting drugs. Most show a considerable placebo effect: in the Intrinsa studies, for instance, some 36% of patients on placebo wanted to continue after the study closed. Maybe P&G should just market the placebo, Nissen quipped during the panel meeting. Talking about a sexual problem and deciding to tackle it might have a therapeutic effect by itself, researchers say.” These data were contained within the briefing materials presented by P&G Pharmaceuticals to the FDA in December 2004.

The importance of the psychological aspect of decreased sexual desire component of FSD can also be understood in Dr. Ngunt's report in the 2005 Journal of Sexual and Marital Therapy titled, “Androgens status in healthy premenopausal women with loss of libido.” After measuring each woman's serum testosterone and comparing this to validated tests to measure reported decreased desire, Dr. Ngunt concluded “loss of libido in otherwise healthy women may be related to relationship problem, depression, psychological factors, and sexual dysfunction in the partner, but do not appear to be related to androgen status.” The psychological basis of treatment of the decreased sexual desire seems to be twofold. First, address the problem and do something to increase desire, like the placebo transdermal patches in the Intrinsa clinical trials. Secondarily, to create a satisfying sexual experience. If a woman experiences a positive sexual intimacy by achieving orgasm with each and every episode of intimacy, she will more likely desire more positive experiences. This can be more understood by referring to the definition of desire as supplied by Webster's Encyclopedic Unabridged Dictionary. Desire is defined as “1. to wish or long for; crave; want. 2. to express a wish to obtain; ask for; request. 3. a longing or craving, as for something that brings satisfaction or enjoyment. 4. an expressed request; request. 5. something desired. 6. sexual appetite or a sexual urge.”

The difference between physiological based treatment of decreased desire with transdermal testosterone, and psychologically based treatment of decreased desire with the introduction of more positive experiences by increased arousal and orgasms, is temporal. After applying the transdermal testosterone patch, a woman can expect an immediate increase in her serum testosterone level, If decreased sexual desire is due to decreased testosterone, improvement in sexual desire should be expected within one or two weeks. If the decreased sexual desire is more psychologically based because of the lack of positive sexually satisfying experiences, or other psychological reasons, the improvement in sexual desire should occur more stepwise and gradually over several months. In fact, this is what was subjectively reported by women in a clinical trial of topical menthol/L-arginine. A number of women, all with very decreased serum testosterone due to oral contraceptives reported a renewed interest in intimacy and an increased sexual desire after six to eight uses of the topical clitoral menthol/L-arginine. One study participant reported, “the orgasms were so intense, I wanted to have sex more of ten.”

### DESCRIPTION OF THE PRESENT INVENTION

The method of the topical clitoral application of menthol, L-arginine, and vardenafil as described in patent application ______ to be used as an effective treatment of any one, a combination of several, or all of the FDA defined components of Female Sexual Dysfunction. The components defined by the FDA Guidance to Industry are:
decreased sexual desire
decreased sexual arousal
dyspareunia
decreased ability to achieve orgasm or inability to achieve orgasm.
   The intent of this invention is to use the topical clitoral combination of menthol/L-arginine/vardenafil as an effective first line therapy for any one, any combination of, or all components of Female Sexual Dysfunction. If not optimally effective, a second effective therapeutic modality such as testosterone therapy and possibly even a third modality such as topical alprostadil as described in patent application Ser. No. 10/989,978 could be added to the topical menthol/L-arginine/vardenafil therapy, as doubled or triple therapy for FSD.

The invention thus comprises a compound for the topical manual application directly to the female clitoris containing Vardenafil, L-arginine and; Menthol or any cooling agent listed below, having a concentration of less than 0.5% and greater than 0.01%; Wherein the cooling agent includes Menthol; wherein the cooling agent includes peppermint oil; wherein the cooling agent includes cornmint oil; wherein the cooling agent includes Eucalypus oil; wherein the cooling agent includes Citronella oil wherein the cooling agent includes Camphor oil; wherein the cooling agent includes Cinnamon oil; wherein the cooling agent essentially comprises Menthol; wherein the cooling agent essentially comprises peppermint oil; wherein the cooling agent essentially comprises cornmint oil; wherein the cooling agent essentially comprises Eucalyptus oil; wherein the cooling agent essentially comprises Citronella oil; wherein the cooling agent essentially comprises Camphor oil; wherein the cooling agent essentially comprises Cinnamon oil; wherein the cooling agent includes a menthol analog or derivative with cooling properties; Menthol Analogs and Derivatives: (+)-neo-Menthol; Menthone; (+)-iso-Menthone; Menthyl acetate; Menthyl isovalerate; (−)-Menthyl lactate; para-menth-1-en-3ol; Piperitone; (−)-Menthol ethylene glycol carbonate; (−)-Menthol 1-and 2-propylene glycol carbonate; (−)-Menthone 1,2-glycerol ketal; (+)-Menthone 1,2-glycerol ketal; and mono-Menthyl succinate.

The invention thus comprises: a method to treat sexual arousal disorder, a subset of Female Sexual Dysfunction, by the application of the topical compound described hereinabove; a method to treat hypoactive sexual desire disorder, a subset of Female Sexual Dysfunction, by the application of the topical compound described hereinabove; a method to treat dyspareunia, a subset of Female Sexual Dysfunction, by the application of the topical compound described hereinabove; a method to treat anorgasmia, a subset of Female Sexual Dysfunction, by the application of the topical compound described hereinabove; the use of the topical compound described hereinabove for treating multiple or all etiologies of Female Sexual Dysfunction; the use of the topical compound described hereinabove for treating multiple or all etiologies of Female Sexual Dysfunction in women that show personal distress; the use of the topical compound described hereinabove for treating one, multiple or all etiologies of Female Sexual Dysfunction in women that do not show personal distress; a method for treating Female Sexual Dysfunction where the topical compound described hereinabove is not systemically absorbed; a method for treating Female Sexual Dysfunction where the topical compound described hereinabove is only partially systemically absorbed; a method for treating Female Sexual Dysfunction where the topical compound described hereinabove is fully systemically absorbed; a method for treating Female Sexual Dysfunction where the topical compound described hereinabove is used episodically, before and with sexual activity; a method for treating Female Sexual Dysfunction where the topical compound described hereinabove is used daily; a method for treating Female Sexual Dysfunction where the topical compound described hereinabove is used concurrently with the use of testosterone, a method for treating Female Sexual Dysfunction where the topical compound listed hereinabove is used concurrently with an oral prohormone of testosterone (DHEA); a method for treating Female Sexual Dysfunction where the topical compound listed hereinabove is used concurrently with a topical alprostadil preparation; a method for treating Female Sexual Dysfunction where the topical compound described hereinabove is used concurrently with the use of topical estrogens; a method for treating Female Sexual Dysfunction where the topical compound described hereinabove is used concurrently with the use of topical phytoestrogens (plant derived)

1. A compound for the topical manual application directly to the female clitoris containing Vardenafil, L-arginine and;
Menthol or any cooling agent listed below, having a concentration of less than 0.5% and greater than 0.01%;
a) wherein said cooling agent includes menthol
b) wherein said cooling agent includes peppermint oil:
c) wherein said cooling agent includes cornmint oil.
d) wherein said cooling agent includes Eucalypus oil.
e) wherein said cooling agent includes Citronella oil.
f) wherein said cooling agent includes Camphor oil.
g) wherein said cooling agent includes Cinnamon oil.
h) Wherein said cooling agent essentially comprises Menthol
i) wherein said cooling agent essentially comprises peppermint oil.
j) wherein said cooling agent essentially comprises cornmiint oil.
k) wherein said cooling agent essentially comprises Eucalyptus' oil.
l) wherein said cooling agent essentially comprises Citronella oil.
m) wherein said cooling agent essentially comprises Camphor oil.
n) wherein said cooling agent essentially comprises Cinnamon oil.
o) wherein said cooling agent includes a menthol analog or derivative with cooling properties;
Menthol Analogs and Derivatives
(+)-neo-Menthol
Menthone
(+)-iso-Menthone
Menthyl acetate
Menthyl isovalerate
(−)-Menthyl lactate
para-menth-1-en-3ol
Piperitone
(−)-Menthol ethylene glycol carbonate
(−)-Menthol 1-and 2-propylene glycol carbonate
(−)-Menthone 1,2-glycerol ketal
(+)-Menthone 1,2-glycerol ketal
mono-Menthyl succinate

2. A method to treat sexual arousal disorder, a subset of Female Sexual Dysfunction, by the application of the topical compound described in claim 1

3. A method to treat hypoactive sexual desire disorder, a subset of Female Sexual Dysfunction, by the application of the topical compound described in claim 1

4. A method to treat dyspareunia, a subset of Female Sexual Dysfunction, by the application of the topical compound described in claim 1

5. A method to treat anorgasmia, a subset of Female Sexual Dysfunction, by the application of the topical compound described in claim 1

6. The use of the topical compound described in claim 1 for treating multiple or all etiologies of Female Sexual Dysfunction

7. The use of the topical compound described in claim 1 for treating multiple or all etiologies of Female Sexual Dysfunction in women that show personal distress

8. The use of the topical compound described in claim 1 for treating one, multiple or all etiologies of Female Sexual Dysfunction in women that do not show personal distress

9. A method for treating Female Sexual Dysfunction where the topical compound described in claim 1 is not systemically absorbed

10. A method for treating Female Sexual Dysfunction where the topical compound described in claim 1 is only partially systemically absorbed

11. A method for treating Female Sexual Dysfunction where the topical compound described in claim 1 is fully systemically absorbed

12. A method for treating Female Sexual Dysfunction where the topical compound described in claim 1 is used episodically, before and with sexual activity

13. A method for treating Female Sexual Dysfunction where the topical compound described in claim 1 is used daily

14. A method for treating Female Sexual Dysfunction where the topical compound described in claim 1 is used concurrently with the use of testosterone

15. A method for treating Female Sexual Dysfunction where the topical compound listed in claim 1 is used concurrently with an oral prohormone of testosterone (DHEA)

16. A method for treating Female Sexual Dysfunction where the topical compound listed in claim 1 is used concurrently with a topical alprostadil preparation

17. A method for treating Female Sexual Dysfunction where the topical compound described in claim 1 is used concurrently with the use of topical estrogens

18. A method for treating Female Sexual Dysfunction where the topical compound described in claim 1 is used concurrently with the use of topical phytoestrogens (plant derived)
